SEQUENCE LISTING (45 sequences)

SEQ ID NO: 1 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 2 - protein, 215 aa, Mus musculus
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEDDEEDEEDEEEEEEEEDEDEEEDDDDE

SEQ ID NO: 3 - protein, 209 aa, Homo sapiens
MGKGDPNKPRGKMSSYAFFVQTCREEHKKKHPDSSVNFAEFSKKCSERWKTMSAKEKSKFEDMAKSDKARYDREMKNYVPPKGDKKGKKKDPNAPKRPPSAFFLFCSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 4 - protein, 54 aa, Homo sapiens
PDASVNFSEFSKKCSERWKTMSAKEKGKFEDMAKADKARYEREMKTYIPPKGET

SEQ ID NO: 5 - protein, 69 aa, Homo sapiens
NAPKRPPSAFFLFCSEYRPKIKGEHPGLSIGDVAKKLGEMWNNTAADDKQPYEKKAAKLKEKYEKDIAA

SEQ ID NO: 6 - DNA, 22 bp, Homo sapiens
gatgggcaaaggagatcctaag

SEQ ID NO: 7 - DNA, 29 bp, Homo sapiens
gcggccgcttattcatcatcatcatcttc

SEQ ID NO: 8 - DNA, 22 bp, Homo sapiens
gatgggcaaaggagatcctaag

SEQ ID NO: 9 - DNA, 32 bp, Homo sapiens
gcggccgctcacttgcttttttcagccttgac

SEQ ID NO: 10 - DNA, 21 bp, Homo sapiens
gagcataagaagaagcaccca

SEQ ID NO: 11 - DNA, 32 bp, Homo sapiens; identical to SEQ ID NO: 9

SEQ ID NO: 12 - DNA, 24 bp, Homo sapiens
aagttcaaggatcccaatgcaaag

SEQ ID NO: 13 - DNA, 32 bp, Homo sapiens
gcggccgctcaatatgcagctatatccttttc

SEQ ID NO: 14 - DNA, 22 bp, Homo sapiens
gatgggcaaaggagatcctaag

SEQ ID NO: 15 - DNA, 24 bp, Homo sapiens
tcacttttttgtctcccctttggg

SEQ ID NO: 16 - protein, 20 aa, Homo sapiens
NAPKRPPSAFFLFCSEYRPK

SEQ ID NO: 17 - protein, 74 aa, Homo sapiens
FKDPNAPKRPPSAFFLFCSEYRPKIKGEHPGLSIGDVAKKLGEMWNNTAADDKQPYEKKAAKLKEKYEKDIAAY

SEQ ID NO: 18 - protein, 216 aa, Homo sapiens [sequence omitted]

SEQ ID NO: 19 - protein, 182 aa, Homo sapiens
MGKGDPKKPTGKMSSYAFFVQTCREEHKKKHPDASVNFSEFSKKCSERWKTMSAKEKGKFEDMAKADKARYEREMKTYIPPKGETKKKFKDPNAPKRLPSAFFLFCSEYRPKIKGEHPGLSIGDVAKKLGEMWNNTAADDKQPYEKKAAKLKEKYEKDIAAYRAKGKPDAAKKGVVKAEKSK

SEQ ID NO: 20 - protein, 74 aa, Homo sapiens
FKDPNAPKRLPSAFFLFCSEYRPKIKGEHPGLSIGDVAKKLGEMWNNTAADDKQPYEKKAAKLKEKYEKDIAAY

SEQ ID NO: 21 - protein, 85 aa, Homo sapiens
MGKGDPKKPTGKMSSYAFFVQTCREEHKKKHPDASVNFSEFSKKCSERWKTMSAKEKGKFEDMAKADKARYEREMKTYIPPKGET

SEQ ID NO: 22 - protein, 77 aa, Homo sapiens
PTGKMSSYAFFVQTCREEHKKKHPDASVNFSEFSKKCSERWKTMSAKEKGKFEDMAKADKARYEREMKTYIPPKGET

SEQ ID NO: 23 - protein, 20 aa, Homo sapiens
FKDPNAPKRLPSAFFLFCSE

SEQ ID NO: 24 - protein, 216 aa, Homo sapiens [sequence omitted]

SEQ ID NO: 25 - protein, 211 aa, Homo sapiens
MGKGDPKKPRGKMSSYAFFVQTCREEHKKKHSDASVNFSEFSNKCSERWKTMSAKEKGKFEDMAKADKTHYERQMKTYIPPKGETKKKFKDPNAPKRPPSAFFLFCSEYHPKIKGEHPGLSIGDVAKKLGEMWNNTAADDKQPGEKKAAKLKEKYEKDIAAYQAKGKPEAAKKGVVKAEKSKKKKEEEEDEEDEEDEEEEDEEDEEDDDDE

SEQ ID NO: 26 - protein, 188 aa, Homo sapiens
MGKGDPKKPRGKMSSYAFFVQTCREECKKKHPDASVNFSEFSKKCSERWKAMSAKDKGKFEDMAKVDKDRYEREMKTYIPPKGETKKKFEDSNAPKRPPSAFLLFCSEYCPKIKGEHPGLPISDVAKKLVEMWNNTFADDKQLCEKKAAKLKEKYKKDTATYRAKGKPDAAKKGVVKAEKSKKKKEEE

SEQ ID NO: 27 - protein, 205 aa, Homo sapiens [sequence omitted]

SEQ ID NO: 28 - protein, 80 aa, Homo sapiens
MGKGDPKKPRGKMSSCAFFVQTCWEEHKKQYPDASINFSEFSQKCPETWKTTIAKEKGKFEDMPKADKAHYEREMKTYIP

SEQ ID NO: 29 - protein, 80 aa, Homo sapiens
KQRGKMPSYVFCVQTCPEERKKKHPDASVNFSEFSKKCLVRGKTMSAKEKGQFEAMARADKARYEREMKTYIPPKGETKK

SEQ ID NO: 30 - protein, 86 aa, Homo sapiens
MGKRDPKQPRGKMSSYAFFVQTAQEEHKKKQLDASVSFSEFSKNCSERWKTMSVKEKGKFEDMAKADKACYEREMKIYPYLKGRQK

SEQ ID NO: 31 - protein, 70 aa, Homo sapiens
MGKGDPKKPREKMPSYAFFVQTCREAHKNKHPDASVNSSEFSKKCSERWKTMPTKQKGKFEDMAKADRAH

SEQ ID NO: 32 - DNA, 648 bp, Homo sapiens
atgggcaaaggagatcctaagaagccgacaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcttccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatatagctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagaagatgaagaagaagatgatgatgatgaa

SEQ ID NO: 33 - DNA, 633 bp, Homo sapiens
atgggcaaaggagatcctaagaagccgagaggcaaaatgtcatcatatgcattttttgtgcaaacttgtcgggaggagcataagaagaagcactcagatgcttcagtcaacttctcagagttttctaacaagtgctcagagaggtggaagaccatgtctgctaaagagaaaggaaaatttgaggatatggcaaaggcggacaagacccattatgaaagacaaatgaaaacctatatccctcccaaaggggagacaaaaaagaagttcaaggatcccaatgcacccaagaggcctccttcggccttcttcctgttctgctctgagtatcacccaaaaatcaaaggagaacatcctggcctgtccattggtgatgttgcgaagaaactgggagagatgtggaataacactgctgcagatgacaagcagcctggtgaaaagaaggctgcgaagctgaaggaaaaatacgaaaaggatattgctgcatatcaagctaaaggaaagcctgaggcagcaaaaaagggagttgtcaaagctgaaaaaagcaagaaaaagaaggaagaggaggaagatgaggaagatgaagaggatgaggaggaggaagatgaagaagatgaagaagatgatgatgatgaa

SEQ ID NO: 34 - DNA, 564 bp, Homo sapiens
atgggcaaaggagaccctaagaagccgagaggcaaaatgtcatcatatgcattttttgtgcaaacttgtcgggaggagtgtaagaagaagcacccagatgcttcagtcaacttctcagagttttctaagaagtgctcagagaggtggaaggccatgtctgctaaagataaaggaaaatttgaagatatggcaaaggtggacaaagaccgttatgaaagagaaatgaaaacctatatccctcctaaaggggagacaaaaaagaagttcgaggattccaatgcacccaagaggcctccttcggcctttttgctgttctgctctgagtattgcccaaaaatcaaaggagagcatcctggcctgcctattagcgatgttgcaaagaaactggtagagatgtggaataacacttttgcagatgacaagcagctttgtgaaaagaaggctgcaaagctgaaggaaaaatacaaaaaggatacagctacatatcgagctaaaggaaagcctgatgcagcaaaaaagggagttgtcaaggctgaaaaaagcaagaaaaagaaggaagaggag

SEQ ID NO: 35 - DNA, 615 bp, Homo sapiens
atggacaaagcagatcctaagaagctgagaggtgaaatgttatcatatgcattttttgtgcaaacttgtcaggaggagcataagaagaagaacccagatgcttcagtcaagttctcagagtttttaaagaagtgctcagagacatggaagaccatttttgctaaagagaaaggaaaatttgaagatatggcaaaggcggacaaggcccattatgaaagagaaatgaaaacctatatccctcctaaaggggagaaaaaaaagaagttcaaggatcccaatgcacccaagaggcctcctttggcctttttcctgttctgctctgagtatcgcccaaaaatcaaaggagaacatcctggcctgtccattgatgatgttgtgaagaaactggcagggatgtggaataacaccgctgcagctgacaagcagttttatgaaaagaaggctgcaaagctgaaggaaaaatacaaaaaggatattgctgcatatcgagctaaaggaaagcctaattcagcaaaaaagagagttgtcaaggctgaaaaaagcaagaaaaagaaggaagaggaagaagatgaagaggatgaacaagaggaggaaaatgaagaagatgatgataaa

SEQ ID NO: 36 - DNA, 240 bp, Homo sapiens
atgggcaaaggagatcctaagaagccgagaggcaaaatgtcatcatgtgcattttttgtgcaaacttgttgggaggagcataagaagcagtacccagatgcttcaatcaacttctcagagttttctcagaagtgcccagagacgtggaagaccacgattgctaaagagaaaggaaaatttgaagatatgccaaaggcagacaaggcccattatgaaagagaaatgaaaacctatataccc

SEQ ID NO: 37 - DNA, 240 bp, Homo sapiens
aaacagagaggcaaaatgccatcgtatgtattttgtgtgcaaacttgtccggaggagcgtaagaagaaacacccagatgcttcagtcaacttctcagagttttctaagaagtgcttagtgagggggaagaccatgtctgctaaagagaaaggacaatttgaagctatggcaagggcagacaaggcccgttacgaaagagaaatgaaaacatatatccctcctaaaggggagacaaaaaaa

SEQ ID NO: 38 - DNA, 258 bp, Homo sapiens
atgggcaaaagagaccctaagcagccaagaggcaaaatgtcatcatatgcattttttgtgcaaactgctcaggaggagcacaagaagaaacaactagatgcttcagtcagtttctcagagttttctaagaactgctcagagaggtggaagaccatgtctgttaaagagaaaggaaaatttgaagacatggcaaaggcagacaaggcctgttatgaaagagaaatgaaaatatatccctacttaaaggggagacaaaaa

SEQ ID NO: 39 - DNA, 211 bp, Homo sapiens
atgggcaaaggagaccctaagaagccaagagagaaaatgccatcatatgcattttttgtgcaaacttgtagggaggcacataagaacaaacatccagatgcttcagtcaactcctcagagttttctaagaagtgctcagagaggtggaagaccatgcctactaaacagaaaggaaaattcgaagatatggcaaaggcagacagggcccata

SEQ ID NO: 40 - protein, 74 aa, Homo sapiens
KKDPNAPKRPPSAFFLFCSEHRPKIKSEHPGLSIGDTAKKLGEMWSEQSAKDKQPYEQKAAKLKEKYEKDIAAY

SEQ ID NO: 41 - protein, 74 aa, Homo sapiens; identical to SEQ ID NO: 20

SEQ ID NO: 42 - protein, 74 aa, Homo sapiens
FKDPNAPKRPPSAFFLFCSEYHPKIKGEHPGLSIGDVAKKLGEMWNNTAADDKQPGEKKAAKLKEKYEKDIAAY

SEQ ID NO: 43 - protein, 74 aa, Homo sapiens
FKDSNAPKRPPSAFLLFCSEYCPKIKGEHPGLPISDVAKKLVEMWNNTFADDKQLCEKKAAKLKEKYKKDTATY

SEQ ID NO: 44 - protein, 74 aa, Homo sapiens
FKDPNAPKRPPSAFFLFCSEYRPKIKGEHPGLSIGDVVKKLAGMWNNTAAADKQFYEKKAAKLKEKYKKDIAAY

SEQ ID NO: 45 - protein, 92 aa, Homo sapiens
FKDPNAPKRPPSAFFLFCSEYRPKIKGEHPGLSIGDVAKKLGEMWNNTAADDKQPYEKKAAKLKEKYEKDIAAYRAKGKPDAAKKGVVKAEK

(19) United States
(12) Patent Application Publication
(10) Pub. No.: US 2006/0121047 A1
(43) Pub. Date: Jun. 8, 2006

(54) Use of hmgb polypetides for increasing immune responses

(76) Inventor: Kevin J. Tracey, Old Greenwich, CT (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/535,267

(22) PCT Filed: Nov. 19, 2003
(86) PCT No.: PCT/US03/36975

§ 371 Date: Nov. 22, 2005

Related U.S. Application Data
(60) Provisional application No. 60/427,848, filed on Nov. 20, 2002.

(51) Int. Cl. A61K 48/00; A61K 39/00; A61K 31/739; A61K 31/4745

(52) U.S. Cl. 424/185.1; 514/54; 514/44; 514/292

(57) ABSTRACT

The present invention features polypeptides comprising an HMGB B box or a functional variant thereof that are useful for stimulating or increasing an immune response in an individual. Such polypeptides can be used in vaccine formulations and in cancer therapies.

### RELATED APPLICATION

This application claims the benefit of U.S. Provisional Application No. 60/427,848, filed Nov. 20, 2002. The entire teachings of the above application are incorporated herein by reference.

### GOVERNMENT SUPPORT

The invention was supported, in whole or in part, by a grant RO1 GM 57226 from the National Institutes of Health. The Government has certain rights in the invention.

### BACKGROUND OF THE INVENTION

The immune system functions to destroy or neutralize foreign matter. Immune responses protect against infection by microbes, including viruses, bacteria, fungi, and other parasites. In addition, the immune system functions to destroy the body's own cells that have become abnormal, for example, cancer cells, and cells that are old and no longer useful to the body, such as erythrocytes.

Manipulation of the immune system is one way in which a therapeutic or protective immune response can be mounted, and a number of diseases can be treated through manipulation of the immune system. Current therapies for treating immunological disorders include anti-inflammatory agents, for example, corticosteroids, cytotoxic agents, agents that modulate signaling events within the immune system, and antibodies.

There are many diseases in which the action of the immune system is inadequate. Therefore, there is a need for treatments of these diseases.

### SUMMARY OF THE INVENTION

It has been found that HMGB polypeptides, as well as polypeptides comprising an HMGB B box or a functional variant thereof (collectively termed “HMGB B boxes”) are useful for stimulating cytokine activity from cells administered such polypeptides. Thus, HMGB polypeptides and polypeptides comprising an HMGB B box can be used to increase an immune response in an individual and to treat a number of diseases for which an increased immune response is desired. Examples of conditions that can be treated using the reagents and methods as described herein include cancer and viral infections, including HIV/AIDS, allergic disease, and asthma. HMGB B boxes and functional variants described herein can also be used as part of a vaccine, in which an immune response is desired to prevent, ameliorate, or treat an infectious disease.

Accordingly, in one aspect, the invention features a pharmaceutical composition comprising an HMGB polypeptide or a functional fragment or variant thereof (collectively termed “HMGB polypeptides”), or an HMGB B box or a functional variant thereof (collectively termed “HMGB B boxes”), in an amount sufficient to treat a disease or condition in which an increase in an immune response in an individual administered the pharmaceutical composition is desired. In one embodiment, the pharmaceutical composition further comprises a vaccine.

In another aspect, the invention features an antibody attached to a polypeptide comprising an HMGB polypeptide or a functional fragment or variant thereof or an HMGB B box or a functional variant thereof. In one embodiment, the antibody is in a pharmaceutically acceptable carrier.

In another embodiment, the invention features a method of stimulating or increasing an immune response in an individual in need of immunostimulation, the method comprising administering to the individual a polypeptide comprising an HMGB polypeptide or a functional fragment or variant thereof or an HMGB B box or a functional variant thereof. In one embodiment, the individual is being treated for cancer. In another embodiment, the polypeptide is attached to an antibody specific to a target site in the individual in need of immunostimulation. In another embodiment, the polypeptide is co-administered with a vaccine. In another embodiment, the polypeptide is in a pharmaceutically acceptable carrier.

In another aspect, the invention features a method of treating cancer in an individual, the method comprising administering to the individual a therapeutically effective amount of a polypeptide comprising an HMGB polypeptide or a functional fragment or variant thereof or an HMGB B box or a functional variant thereof. In one embodiment, the individual is being treated for cancer. In another embodiment the polypeptide is attached to an antibody specific to a target site in the individual in need of immunostimulation. In another embodiment, the polypeptide is co-administered with a vaccine. In another embodiment, the polypeptide is in a pharmaceutically acceptable carrier.

### BRIEF DESCRIPTION OF THE DRAWINGS

FIG. 1 is a schematic representation of HMG1 mutants and their activity in TNF release (pg/ml).

FIG. 2A is a histogram showing the effect of 0 μg/ml, 0.01 μg/ml, 0.1 μg/ml, 1 μg/ml or 10 μg/ml of B box on TNF release (pg/ml) in RAW 264.7 cells.

FIG. 2B is a histogram showing the effect of 0 μg/ml, 0.01 μg/ml, 0.1 μg/ml, 1 μg/ml or 10 μg/ml of B box on IL-1β release (pg/ml) in RAW 264.7 cells.

FIG. 2C is a histogram showing the effect of 0 μg/ml, 0.01 μg/ml, 0.1 μg/ml, 1 μg/ml or 10 μg/ml of B box on IL-6 release (pg/ml) in RAW 264.7 cells.

FIG. 2D a scanned image of a blot of an RNAse protection assay, showing the effect of B box (at 0 hours, 4 hours, 8 hours, or 24 hours after administration) or vector alone (at 4 hours after administration) on TNF mRNA expression in RAW 264.7 cells.

FIG. 2E is a histogram of the effect of HMG1 B box on TNF protein release (pg/ml) from RAW 264.7 cells at 0 hours, 4 hours, 8 hours, 24 hours, 32 hours or 48 hours after administration.

FIG. 2F is a histogram of the effect of vector on TNF protein release (pg/ml) from RAW 264.7 cells at 0 hours, 4 hours, 8 hours, 24 hours, 32 hours or 48 hours after administration.

FIG. 3 is a schematic representation of HMG1 B box mutants and their activity in TNF release (pg/ml).

FIG. 4A is a scanned image of a hematoxylin and eosin stained kidney section obtained from an untreated mouse.

FIG. 4B is a scanned image of a hematoxylin and eosin stained kidney section obtained from a mouse administered HMG1 B box.

FIG. 4C is a scanned image of a hematoxylin and eosin stained myocardium section obtained from an untreated mouse.

FIG. 4D is a scanned image of a hematoxylin and eosin stained myocardium section obtained from a mouse administered HMG1 B box.

FIG. 4E is a scanned image of a hematoxylin and eosin stained lung section obtained from an untreated mouse.

FIG. 4F is a scanned image of a hematoxylin and eosin stained lung section obtained from a mouse administered HMG1 B box.

FIG. 4G is a scanned image of a hematoxylin and eosin stained liver section obtained from an untreated mouse.

FIG. 4H is a scanned image of a hematoxylin and eosin stained liver section obtained from a mouse administered HMG1 B box.

FIG. 4I is a scanned image of a hematoxylin and eosin stained liver section (high magnification) obtained from an untreated mouse.

FIG. 4J is a scanned image of a hematoxylin and eosin stained liver section (high magnification) obtained from a mouse administered HMG B box.

FIG. 5A is the amino acid sequence of a human HMG1 polypeptide (SEQ ID NO: 1).

FIG. 5B is the amino acid sequence of rat and mouse HMG1 (SEQ ID NO: 2).

FIG. 5C is the amino acid sequence of human HMG2 (SEQ ID NO: 3).

FIG. 5D is the amino acid sequence of a human, mouse, and rat HMG1 A box polypeptide (SEQ ID NO: 4).

FIG. 5E is the amino acid sequence of a human, mouse, and rat HMG1 B box polypeptide (SEQ ID NO: 5).

FIG. 5F is the nucleic acid sequence of a forward primer for human HMG1 (SEQ ID NO: 6).

FIG. 5G is the nucleic acid sequence of a reverse primer for human HMG1 (SEQ ID NO: 7).

FIG. 5H is the nucleic acid sequence of a forward primer for the carboxy terminus mutant of human HMG1 (SEQ ID NO: 8).

FIG. 5I is the nucleic acid sequence of a reverse primer for the carboxy terminus mutant of human HMG1 (SEQ ID NO: 9).

FIG. 5J is the nucleic acid sequence of a forward primer for the amino terminus plus B box mutant of human HMG1 (SEQ ID NO: 10).

FIG. 5K is the nucleic acid sequence of a reverse primer for the amino terminus plus B box mutant of human HMG1 (SEQ ID NO: 11).

FIG. 5L is the nucleic acid sequence of a forward primer for a B box mutant of human HMG1 (SEQ ID NO: 12).

FIG. 5M is the nucleic acid sequence of a reverse primer for a B box mutant of human HMG1 (SEQ ID NO: 13).

FIG. 5N is the nucleic acid sequence of a forward primer for the amino terminus plus A box mutant of human HMG1 (SEQ ID NO: 14).

FIG. 5O is the nucleic acid sequence of a reverse primer for the amino terminus plus A box mutant of human HMG1 (SEQ ID NO: 15).

FIG. 6 is a sequence alignment of HMG1 polypeptide sequence from rat (SEQ ID NO:2), mouse (SEQ ID NO:2), and human (SEQ ID NO: 18).

FIG. 7A is the nucleic acid sequence of HMG1L5 (formerly HMG1L10) (SEQ ID NO: 32) encoding an HMGB polypeptide.

FIG. 7B is the polypeptide sequence of HMG1L5 (formerly HMG1L10) (SEQ ID NO: 24) encoding an HMGB polypeptide.

FIG. 7C is the nucleic acid sequence of HMG1L1 (SEQ ID NO: 33) encoding an HMGB polypeptide.

FIG. 7D is the polypeptide sequence of HMG1L1 (SEQ ID NO: 25) encoding an HMGB polypeptide.

FIG. 7E is the nucleic acid sequence of HMG1L4 (SEQ ID NO: 34) encoding an HMGB polypeptide.

FIG. 7F is the polypeptide sequence of HMG1L4 (SEQ ID NO: 26) encoding an HMGB polypeptide.

FIG. 7G is the nucleic acid sequence of the HMG polypeptide sequence of the BAC clone RP11-395A23 (SEQ ID NO: 35).

FIG. 7H is the polypeptide sequence of the HMG polypeptide sequence of the BAC clone RP11-395A23 (SEQ ID NO: 27) encoding an HMGB polypeptide.

FIG. 7I is the nucleic acid sequence of HMG1L9 (SEQ ID NO: 36) encoding an HMGB polypeptide.

FIG. 7J is the polypeptide sequence of HMG1L9 (SEQ ID NO: 28) encoding an HMGB polypeptide.

FIG. 7K is the nucleic acid sequence of LOC122441 (SEQ ID NO: 37) encoding an HMGB polypeptide.

FIG. 7L is the polypeptide sequence of LOC122441 (SEQ ID NO: 29) encoding an HMGB polypeptide.

FIG. 7M is the nucleic acid sequence of LOC139603 (SEQ ED NO: 38) encoding an HMGB polypeptide.

FIG. 7N is the polypeptide sequence of LOC139603 (SEQ ID NO: 30) encoding an HMGB polypeptide.

FIG. 7O is the nucleic acid sequence of HMG1L8 (SEQ ID NO: 39) encoding an HMGB polypeptide.

FIG. 7P is the polypeptide sequence of HMG1L8 (SEQ ID NO: 31) encoding an HMGB polypeptide.

### DETAILED DESCRIPTION OF THE INVENTION

The present invention features HMGB polypeptides and polypeptides comprising an HMGB B box or a functional variant thereof that are useful for stimulating or increasing an immune response in an individual. In one embodiment the polypeptide comprises or consists of a mammalian HMGB B box, for example, a human HMGB B box. Examples of an HMGB B boxes include polypeptides having the sequence of SEQ ID NO: 5, SEQ ID NO: 20, or SEQ ID NO: 45.

As used herein, an “HMGB polypeptide” or an “HMGB protein” is an isolated, substantially pure, or substantially pure and isolated polypeptide that has been separated from components that naturally accompany it or a recombinantly produced polypeptide having the same amino acid sequence, and increases inflammation, and/or increases release of a proinflammatory cytokine from a cell, and/or increases the activity of the inflammatory cytokine cascade. In one embodiment, the HMGB polypeptide has one of the above biological activities. In another embodiment, the HMGB polypeptide has two of the above biological activities. In a third embodiment, the HMGB polypeptide has all three of the above biological activities.

Preferably, the HMGB polypeptide is a mammalian HMGB polypeptide, for example, a human HMGB1 polypeptide. Examples of an HMGB polypeptide include a polypeptide comprising or consisting of the sequence of SEQ ID NO: 1, SEQ ID NO: 2, SEQ ID NO: 3, or SEQ ID NO: 18. Preferably, the HMGB polypeptide contains a B box DNA binding domain and/or an A box DNA binding domain, and/or an acidic carboxyl terminus as described herein. Other examples of HMGB polypeptides are described in GenBank Accession Numbers AAA64970, AAB08987, P07155, AAA20508, S29857, P09429, NP 002119, CAA31110, S02826, U00431, X67668, NP_{—}005333, NM_{—}016957, and J04179, the entire teachings of which are incorporated herein by reference. Additional examples of HMGB polypeptides include, but are not limited to mammalian HMG1 ((HMGB1) as described, for example, in GenBank Accession Number U51677), HMG2 ((HGB2) as described, for example, in GenBank Accession Number M83665), HMG-2A ((HMGB3, HMG-4) as described, for example, in GenBank Accession Numbers NM_{—}005342 and NP_{—}005333), HMG14 (as described, for example, in GenBank Accession Number P05114), HMG17 (as described, for example, in GenBank Accession Number X13546), HMGI (as described, for example, in GenBank Accession Number L17131), and HMGY (as described, for example, in GenBank Accession Number M23618); nonmammalian HMG T1 (as described, for example, in GenBank Accession Number X02666) and HMG T2 (as described, for example, in GenBank Accession Number L32859) (rainbow trout); HMG-X (as described, for example, in GenBank Accession Number D30765) (Xenopus); HMG D (as described, for example, in GenBank Accession Number X71138) and IMG Z (as described, for example, in GenBank Accession Number X71139) (Drosophila); NHP10 protein (HMG protein homolog NIP 1) (as described, for example, in GenBank Accession Number Z48008) (yeast); non-histone chromosomal protein (as described, for example, in GenBank Accession Number 000479) (yeast); HMG 1/2 like protein (as described, for example, in GenBank Accession Number Z11540) (wheat, maize, soybean); upstream binding factor (UBF-1) (as described, for example, in GenBank Accession Number X53390); PMS1 protein homolog 1 (as described, for example, in GenBank Accession Number U13695); single-strand recognition protein (SSRP, structure-specific recognition protein) (as described, for example, in GenBank Accession Number M86737); the HMG homolog TDP-1 (as described, for example, in GenBank Accession Number M74017); mammalian sex-determining region Y protein (SRY, testis-determining factor) (as described, for example, in GenBank Accession Number X53772); fungal proteins: mat-1 (as described, for example, in GenBank Accession Number AB009451), ste 11 (as described, for example, in GenBank Accession Number x53431) and Mc 1; SOX 14 (as described, for example, in GenBank Accession Number AF107043) (as well as SOX 1 (as described, for example, in GenBank Accession Number Y13436), SOX 2 (as described, for example, in GenBank Accession Number Z31560), SOX 3 (as described, for example, in GenBank Accession Number X71135), SOX 6 (as described, for example, in GenBank Accession Number AF309034), SOX 8 (as described, for example, in GenBank Accession Number AF226675), SOX 10 (as described, for example, in GenBank Accession Number AJ001183), SOX 12 (as described, for example, in GenBank Accession Number X73039) and SOX 21 (as described, for example, in GenBank Accession Number AF107044)); lymphoid specific factor (LEF-1)(as described, for example, in GenBank Accession Number X58636); T-cell specific transcription factor (TCF-1)(as described, for example, in GenBank Accession Number X59869); MTT1 (as described, for example, in GenBank Accession Number M62810); and SP100-HMG nuclear autoantigen (as described, for example, in GenBank Accession Number U36501).

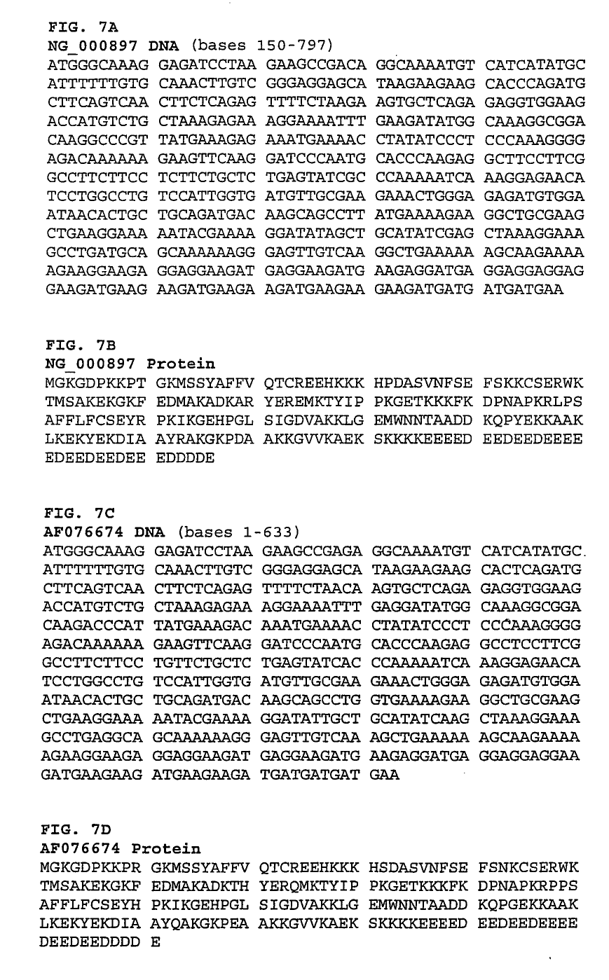

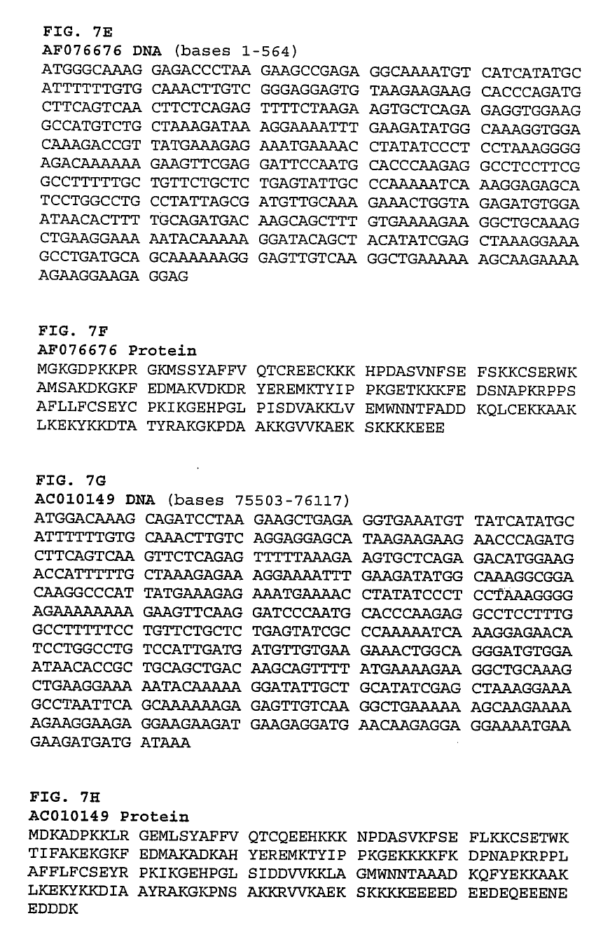

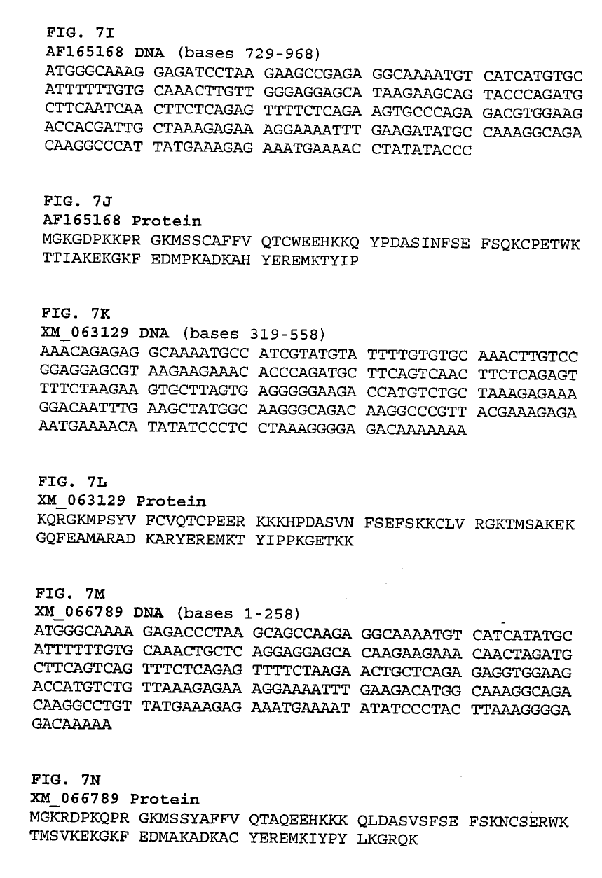

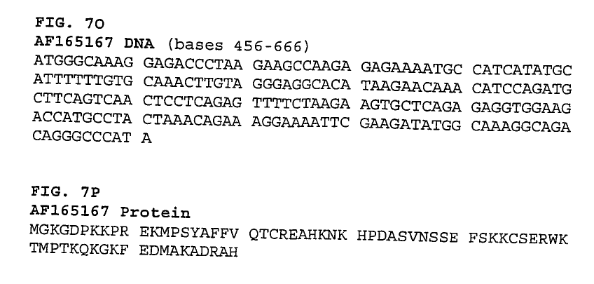

Other examples of HMGB proteins are polypeptides encoded by HMGB nucleic acid sequences having GenBank Accession Numbers NG_{—}000897 (HMG1L5 (formerly HMG1L10)) (and in particular by nucleotides 150-797 of NG_{—}000897, as shown in FIGS. 7A and 7B); AF076674 (HMG1L1) (and in particular by nucleotides 1-633 of AF076674, as shown in FIGS. 7C and 7D; AF076676 (HMG1L4) (and in particular by nucleotides 1-564 of AF076676, as shown in FIGS. 7E and 7F); AC010149 (HMG sequence from BAC clone RP11-395A23) (and in particular by nucleotides 75503-76117 of AC010149), as shown in FIGS. 7G and 7H); AF165168 (HMG1L9) (and in particular by nucleotides 729-968 of AF165168, as shown in FIGS. 7I and 7J); XM_{—}063129 (LOC122441) (and in particular by nucleotides 319-558 of XM_{—}063129, as shown in FIGS. 7K and 7L); XM_{—}066789 (LOC139603) (and in particular by nucleotides 1-258 of XM_{—}066789, as shown in FIGS. 7M and 7N); and AF165167 (HMG1L8) (and in particular by nucleotides 456-666 of AF165167, as shown in FIGS. 70 and 7P).

The HMGB polypeptides of the present invention also encompass sequence variants. Variants include a substantially homologous polypeptide encoded by the same genetic locus in an organism, i.e., an allelic variant, as well as other variants. Variants also encompass polypeptides derived from other genetic loci in an organism, but having substantial homology to a polypeptide encoded by an HMGB nucleic acid molecule, and complements and portions thereof or having substantial homology to a polypeptide encoded by a nucleic acid molecule comprising the nucleotide sequence of an HMGB nucleic acid molecule. Examples of HMGB nucleic acid molecules are known in the art and can be derived from HMGB polypeptides as described herein. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods. Preferably, the HMGB polypeptide has at least 60%, more preferably, at least 70%, 75%, 80%, 85%, or 90%, and most preferably at least 95% sequence identity to a sequence selected from SEQ ID NO:1, SEQ ID NO:2, SEQ ID NO:3, or SEQ ID NO:18, as determined using the BLAST program and parameters described herein and one of more of the biological activities of an HMGB polypeptide (functional variant).

In other embodiments, the present invention is directed to an HMGB polypeptide fragment that has HMGB biological activity (functional fragment). By an “HMGB polypeptide fragment that has HMGB biological activity” or a “biologically active HMGB fragment” is meant a fragment of an HMGB polypeptide that has the activity of an HMGB polypeptide. An example of such an HMGB polypeptide fragment is the HMGB B box, as described herein. Biologically active HMGB fragments can be generated using standard molecular biology techniques and assaying the function of the fragment by determining if the fragment, when administered to a cell increase release of a proinflammatory cytokine from the cell, compared to a suitable control, for example, using methods described herein.

As used herein, an “HMGB B box” also referred to herein as a “B box” is a substantially pure, or substantially pure and isolated polypeptide that has been separated from components that naturally accompany it, and consists of an amino acid sequence that is less than a full length HMGB polypeptide and has one or more of the following biological activities: increasing inflammation, increasing release of a proinflammatory cytokine from a cell, and/or increasing the activity of the inflammatory cytokine cascade. In one embodiment, the HMGB B box polypeptide has one of the above biological activities. In another embodiment, the HMGB B box polypeptide has two of the above biological activities. In a third embodiment, the HMGB B box polypeptide has all three of the above biological activities. Preferably, the HMGB B box has at least 25%, 30%, 40%, 50%, 60%, 70%, 80% or 90% of the biological activity of full length HMG. In another embodiment, the HMGB B box does not comprise an HMGB A box. In another embodiment, the HMGB B box is a fragment of and HMGB polypeptide (i.e., a polypeptide that is about 90%, 80%, 70%, 60%, 50%, 40%, 35%, 30%, 25%, or 20% the length of a full length HMG1 polypeptide). In another embodiment, the HMGB B box comprises or consists of the sequence of SEQ ID NO: 5, SEQ ID NO: 20, SEQ ID NO: 45, or the amino acid sequence in the corresponding region of an HMGB protein in a mammal, but is still less than the full length HMGB polypeptide. An HMGB B box polypeptide is also a recombinantly produced polypeptide having the same amino acid sequence as an HMGB B box polypeptide described above. Preferably, the HMGB B box is a mammalian HMGB B box, for example, a human HMGB1 B box. An HMGB B box often has no more than about 85 amino acids and no fewer than about 4 amino acids.

Examples of polypeptides having B box sequences within them include, but are not limited to HMGB polypeptides described herein. The B box sequences in such polypeptides can be determined and isolated using methods described herein, for example, by sequence comparisons to B boxes described herein and testing for B box biological activity. In particularly preferred embodiments, the B box comprises SEQ ID NO:5, SEQ ED NO:20, or SEQ ID NO:45, which are the sequences (three different lengths) of the human HMGB1 B box, or is a fragment of an HMGB B box that has B box biological activity. For example, a 20 amino acid sequence contained within SEQ ID NO:20 contributes to the function of the B box. This 20 amino acid B-box fragment has the following amino acid sequence: fkdpnapkrl psafflfcse (SEQ ID NO:23). Another example of an HMGB B box biologically active fragment consists of amino acids 1-20 of SEQ ID NO:5 (napkrppsaf flfcseyrpk; SEQ ID NO:16).

Examples of HMGB B box polypeptide sequences include the following sequences: FKDPNAPKRP PSAFFLFCSE YRPKIKGEHP GLSIGDVAKK LGEMWNNTAA DDKQPYEKKA AKLKEKYEKD IAAY (human HMGB1; SEQ ID NO: 17); KKDPNAPKRP PSAFFLFCSE HRPKIKSEHP GLSIGDTAKK LGEMWSEQSA KDKQPYEQKA AKLKEKYEKD IAAY (human HMGB2; SEQ ID NO: 40); FKDPNAPKRL PSAFFLFCSE YRPKIKGEHP GLSIGDVAKK LGEMWNNTAA DDKQPYEKKA AKLKEKYEKD IAAY (HMG1L5 (formerly HMG1L10); SEQ ID NO: 41); FKDPNAPKRP PSAFFLFCSE YHPKIKGEHP GLSIGDVAKK LGEMWNNTAA DDKQPYEKKA AKLKEKYEKD IAAY (HMG1L1; SEQ ID NO: 42); FKDSNAPKRP PSAFLLFCSE YCPKIKGEHP GLPISDVAKK LVEMWNNTFA DDKQLCEKKA AKLKEKYKKD TATY (HMG1L4; SEQ ID NO: 43); FKDPNAPKRP PSAFFLFCSE YRPKIKGEHP GLSIGDVAKK LAGMWNNTAA ADKQFYEKKA AKLKEKYKKD IAAY (HMG sequence from BAC clone RP11-359A23; SEQ ID NO: 44); and FKDPNAPKRP PSAFFLFCSE YRPKIKGEHP GLSIGDVAKK LGEMWNNTAA DDKQPYEKKA AKLKEKYEKD IAAYRAKGKP DAAKKGVVKA EK (human HMGB1 box; SEQ ID NO: 45).

The HMGB B box polypeptides of the invention also encompasses sequence variants that are functional variants, and can be naturally-occurring or non-naturally-occurring. Functional variants include a substantially homologous polypeptide encoded by the same genetic locus in an organism, i.e., an allelic variant, as well as other variants. Functional variants also encompass polypeptides derived from other genetic loci in an organism, but having substantial homology to a polypeptide encoded by an HMGB nucleic acid molecule, and complements and portions thereof, or having substantial homology to a polypeptide encoded by a nucleic acid molecule comprising the nucleotide sequence of an HMGB B box nucleic acid molecule. Examples of HMGB B box nucleic acid molecules are known in the art and can be derived from HMGB B box polypeptides as described herein. Functional variants also include polypeptides substantially homologous or identical to these polypeptides but derived from another organism, i.e., an ortholog. Functional variants also include polypeptides that are substantially homologous or identical to these polypeptides that are produced by chemical synthesis. Functional variants also include polypeptides that are substantially homologous or identical to these polypeptides that are produced by recombinant methods:

Preferably, an HMGB B box polypeptide variant has at least 60%, more preferably, at least 70%, 75%, 80%, 85%, or 90%, and most preferably at least 95% sequence identity to the sequence of an HMGB B box as described herein, for example, the sequence of SEQ ID NO: 5, SEQ ID NO: 20, or SEQ ID NO: 45, as determined using the BLAST program and parameters described herein. Preferably, the HMGB B box consists of the sequence of SEQ ID NO: 5, SEQ ID NO: 20, or SEQ ID NO: 45, or the amino acid sequence in the corresponding region of an HMGB protein in a mammal, and has one or more of the biological activities of an HMGB B box, determined using methods described herein or other methods known in the art.

As used herein, two polypeptides (or a region of the polypeptides) are substantially homologous or identical when the amino acid sequences are at least about 60%, 70%, 75%, 80%, 85%, 90% or 95% or more homologous or identical. The percent identity of two amino acid sequences (or two nucleic acid sequences) can be determined by aligning the sequences for optimal comparison purposes (e.g., gaps can be introduced in the sequence of a first sequence). The amino acids or nucleotides at corresponding positions are then compared, and the percent identity between the two sequences is a function of the number of identical positions shared by the sequences (i.e., % identity=# of identical positions/total # of positions×100). In certain embodiments, the length of the HMGB polypeptide or HMGB B box polypeptide aligned for comparison purposes is at least 30%, preferably, at least 40%, more preferably, at least 60%, and even more preferably, at least 70%, 80%, 90%, or 100% of the length of the reference sequence, for example, those sequence provided herein. The actual comparison of the two sequences can be accomplished by well-known methods, for example, using a mathematical algorithm. A preferred, non-limiting example of such a mathematical algorithm is described in Karlin et al. (Proc. Natl. Acad. Sci. USA, 90:5873-5877, 1993). Such an algorithm is incorporated into the BLASTN and BLASTX programs (version 2.2) as described in Schaffer et al. (Nucleic Acids Res., 29:2994-3005, 2001). When utilizing BLAST and Gapped BLAST programs, the default parameters of the respective programs (e.g., BLASTN) can be used. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Another preferred, non-limiting example of a mathematical algorithm utilized for the comparison of sequences is the algorithm of Myers and Miller, CABIOS (1989). Such an algorithm is incorporated into the ALIGN program (version 2.0), which is part of the GCG (Accelrys, San Diego, Calif.) sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, Comput. Appl. Biosci., 10: 3-5, 1994; and FASTA described in Pearson and Lipman, Proc. Natl. Acad. Sci USA, 85: 2444-2448, 1988.

In another embodiment, the percent identity between two amino acid sequences can be accomplished using the GAP program in the GCG software package (Accelerys) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package (Accelrys), using a gap weight of 50 and a length weight of 3.

HMGB polypeptides of functional fragments or variants thereof (collectively termed “HMGB polypeptides”), or polypeptides comprising an HMGB B box or a functional variant thereof (collectively termed “HMGB B boxes”) can be used in pharmaceutical compositions to stimulate or increase an immune response. As used herein, by an “immune response” is meant a collective and coordinated response to the introduction of a foreign substance in the body, by cells and molecules of the immune system. Cytokines play an important role in mediating immune responses. Thus molecules that stimulate cytokine activity are useful for developing and/or mediating immune responses.

In one embodiment, the pharmaceutical composition comprises the HMGB B box and a vaccine. The vaccine can be administered to a person in need of immunostimulation (i.e., a person who would benefit by mounting or increasing an immune response to an antigen, a tumor cell or a tumor) in order to stimulate an immune response. Examples of vaccines include Hepatitis B Diptheria, Tetanus, Pertussis, Haemoplilus influenzae Type B, Inactivated Polio, Measles, Mumps, Rubella, Varicella, Pneumococcal, Hepatitis A, Influenza, Japanese Encephalitis, Rotavirus, Yellow Fever, Trypanosoma cruzi; and Rabies. If desired, the pharmaceutical composition can further comprise an adjuvant. As used herein, an “adjuvant” is an immunologic reagent that increases an antigenic response. Examples of adjuvants for use in pharmaceuticals include immunostimulatory oligonucleotides, imidazoquinolines (e.g., imiquimod), monophosphoryl lipid A, and detoxified lipopolysaccharide (LPS), as described, for example, by O'Hagan et al. (Biomol. Eng. 18:69-85, 2001)). An example of an immunostimulatory oligonucleotide is an oligonucleotide having ummethylated CpG sequences.

In another example, the pharmaceutical composition comprises an HMGB polypeptide or functional fragment or variant thereof or an HMGB B box polypeptide or functional variant thereof attached to an antibody. The antibody specifically binds a polypeptide, preferably an epitope, or a target site (as determined, for example, by immunoassays, a technique well known in the art for assaying specific antibody-antigen binding) to deliver the HMGB B box polypeptide to the target site in order to stimulate or increase an immune response at the site where the antibody binds. Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, for example, anti-Id antibodies to antibodies of the invention), and epitope-binding fragments of any of the above.

The term “antibody,” as used herein, refers to immunoglobulin molecules and immunologically active portions of immunoglobulin molecules, and more specifically, molecules that contain an antigen binding site that specifically binds an antigen. The immunoglobulin molecules of the invention can be of any type (for example, IgG, IgE, IgM, IgD, IgA and IgY), and of any class (for example, IgG1, IgG2, IgG3, IgG4, IgA1 and IgA2) or subclass of an immunoglobulin molecule.

In one embodiment, the antibodies are antigen-binding antibody fragments and include, without limitation, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, can comprise the variable region(s) alone or in combination with the entirety or a portion of one or more of the following: hinge region, CH1, CH2, and CH3 domains. Also included in the invention are antigen-binding fragments also comprising any combination of variable region(s) with a hinge region, CH1, CH2, and/or CH3 domains.

The antibodies of the invention may be from any animal origin including birds and mammals. Preferably, the antibodies are human, murine, donkey, sheep, rabbit, goat, guinea pig, hamster, horse, or chicken.

As used herein, “human” antibodies include antibodies having the amino acid sequence of a human immunoglobulin and include antibodies produced by human B cells, or isolated from human sera, human immunoglobulin libraries or from animals transgenic for one or more human immunoglobulins and that do not express endogenous immunoglobulins, as described in U.S. Pat. No. 5,939,598 by Kucherlapati et al., for example.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. The term “epitope,” as used herein, refers to a portion of a polypeptide which contacts an antigen-binding site(s) of an antibody or T cell receptor.

The term “target site” as used herein, refers to a polypeptide that is recognized by an antibody and to which the antibody binds. The target site is preferably a site at which delivery or localization of an HMGB B box polypeptide or functional variant thereof is desired. The target site can be in vivo or ex vitro. The target site can be, for example, a polypeptide localized on the surface of a cell or near (e.g., adjacent to) a cell to which delivery of an HMGB B box is desired. In one embodiment the target site is a cancer target site, for example, a cancer cell or a site near a tumor, such that delivery of an HMGB polypeptide to the cancer cell or tumor occurs. In such a case, the antibody may be a tumor-associated antibody (i.e., an antibody that is preferentially or exclusively bound by a cancer cell or tumor).

In one embodiment, the antibody of the present invention, attached to an HMGB B box or functional variant thereof is a tumor-associated antibody that binds to a tumor-associated polypeptide, marker, or antigen at a cancer target site. Tumor-associated polypeptides or markers include, but are not limited to oncofetal antigens, placental antigens, oncogenic or tumor virus-associated antigens, tissue-associated antigens, organ-associated antigens, ectopic hormones and normal antigens or variants thereof. A sub-unit of a tumor-associated marker can also be used to raise antibodies having higher tumor-specificity, e.g., the beta-subunit of human chorionic gonadotropin (HCG), which stimulates the production of antibodies having a greatly reduced cross-reactivity to non-tumor substances. Suitable such marker substances to which specific antibodies may be raised and/or obtained which are useful in the present invention include, but are not limited to, alpha-fetoprotein (AFP), human chorionic gonadotropin (HCG) and/or its beta-subunit (HCG-beta), colon-specific antigen-p (CSAp), prostatic acid phosphatase, pancreatic oncofetal antigen, placental alkaline phosphatase, pregnancy beta₁-globulin, parathormone, calcitonin, tissue polypeptide antigen, T-antigen, beta₂-microglobulin, mammary tumor-associated glycoproteins (MTGP), galactyosyl transferase-II (GT-II), gp-52 viral-associated antigen, ovarian cystadenocarcinoma-associated antigen (OCAA), ovarian tumor-specific antigen (OCA), cervical cancer antigens (CA-58, CCA, TA-4), basic fetoprotein (BFP), terminal deoxynucleotidyl transferase (TdT), cytoplasmic melanoma-associated antigens, human astrocytoma-associated antigen (HAAA), common glioma antigen (CGA), glioembryonic antigen (GEA), glial fibrillary acidic protein (GFA), common meningioma antigen (CMA), ferritin, and tumor angiogenesis factor (TAF).

Antibodies of the present invention may also be described or specified in terms of their cross-reactivity. Antibodies used in the present invention may not display significant cross-reactivity, such that they do not bind any other analog, ortholog, or homolog of a polypeptide of the present invention. Alternatively, antibodies of the invention can bind polypeptides with at least about 95%, 90%, 85%, 80%, 75%, 70%, 65%, 60%, 55%, or 50% identity (as calculated using methods known in the art) to a polypeptide at a target site.

Antibodies of the present invention can also be described or specified in terms of their binding affinity to a polypeptide at a target site. Preferred binding affinities include those with a dissociation constant or Kd less than 5×10⁻⁶ M, 10⁻⁶ M, 5×10⁻⁷ M, 10⁻⁷ M, 5×10⁻⁸M, 10⁻⁹M, 5×10⁻⁹ M, 10⁻⁹ M, 5×10⁻¹⁰M, 5×10⁻¹⁰M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹³ M, 5×10⁻¹⁵ M, and 10⁻¹⁵ M.

Antibodies used in the present invention can act as agonists or antagonists of a polypeptide at a target site. For example, the present invention includes antibodies which disrupt interactions with the polypeptides at the target site either partially or fully. The invention also includes antibodies that do not prevent binding, but prevent activation or activity of the polypeptide. Activation or activity (for example, signaling) may be determined by techniques known in the art. Also included are antibodies that prevent both binding to and activity of a polypeptide at a target site. Likewise included are neutralizing antibodies.

The antibodies used in the invention include derivatives that do not prevent the antibody from recognizing its epitope. For example, but not by way of limitation, the antibody derivatives include antibodies that have been modified, for example, by glycosylation, acetylation, pegylation, phosphorylation, amidation, derivatization by known protecting/blocking groups, or proteolytic cleavage.

The antibodies used in the invention can be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, or the like, to induce the production of sera containing polyclonal antibodies specific for the antigen. Various adjuvants can be used to increase the immunological response, depending on the host species, and include, but are not limited to, Freund's adjuvant (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and corynebacterium parvum. Such adjuvants are well known in the art.

Monoclonal antibodies can be prepared using a wide variety of techniques also known in the art, including hybridoma cell culture, recombinant, and phage display technologies, or a combination thereof. For example, monoclonal antibodies can be produced using hybridoma techniques as is known in the art and taught, for example, in Harlow et al., Antibodies: A Laboratory Manual (Cold Spring Harbor Laboratory Press, 2nd ed. 1988). The term “monoclonal antibody” as used herein is not necessarily limited to antibodies produced through hybridoma technology, but also refers to an antibody that is derived from a single clone, including any eukaryotic, prokaryotic, or phage clone.

Human antibodies are desirable for therapeutic treatment of human patients. These antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes. The transgenic mice are immunized with a selected antigen, for example, all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For a detailed discussion of this technology for producing human antibodies and human monoclonal antibodies and protocols for producing such antibodies, see, for example, PCT publications WO 98/24893; WO 96/34096; WO 96/33735; and U.S. Pat. Nos. 5,413,923; 5,625,126; 5,633,425; 5,569,825; 5,661,016; 5,545,806; 5,814,318; and 5,939,598.

An HMGB polypeptide or HMGB B box polypeptide can be attached, coupled, or conjugated to an antibody using methods known to one of skill in the art. In one embodiment, the polypeptide is covalently attached to the antibody. In another embodiment the polypeptide-antibody conjugate is produced using recombinant methods, and is generated as a fusion protein comprising the polypeptide and the antibody or an antigen binding fragment of an antibody. Alternatively, the polypeptide can be chemically crosslinked to the antibody. If desired, spacers or linkers (for example, those available from Pierce Chemical Company) may be used to attached the polypeptide to the linker. Methods for attaching a polypeptide to an antibody are described, for example, by Jeanson et al. (J. Immunol Methods 111:261-270, 1988); and Zarling et al. (Int. J. Immunopharmacol. 13 Suppl 1:63-68-1991). Reactive groups that can be targeted by coupling agents include primary amines, sulfhydryls, and carbonyls.

The compositions of the invention can be administered alone or in combination with other therapeutic agents. Therapeutic agents that can be administered in combination with the compositions of the invention, include but are not limited to chemotherapeutic agents, antibiotics, steroidal and non-steroidal anti-inflammatories, conventional immunotherapeutic agents, cytokines and/or growth factors. Combinations may be administered either concomitantly, for example, as an admixture, separately but simultaneously or concurrently; or sequentially.

In another embodiment, compositions of the invention are administered in combination with a chemotherapeutic agent. Chemotherapeutic agents that may be administered with the compositions of the invention include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, taxol, and etoposide).

In an additional embodiment, the compositions of the invention may be administered in combination with cytokines. Cytokines that may be administered with the compositions of the invention include, but are not limited to, IL2, IL3, IL4, IL5, IL6, IL7, IL10, IL12, IL13, IL15, anti-CD40, CD40L, IFN-gamma and TNF-alpha.

In additional embodiments, the compositions of the invention are administered in combination with other therapeutic or prophylactic regimens, such as, for example, radiation therapy.

As described herein, the compositions comprising HMGB polypeptides or functional fragments or variants thereof or HMGB B box polypeptides or functional variants thereof can be formulated in a pharmaceutically acceptable carrier. The pharmaceutically acceptable carrier included with the polypeptide in these compositions is chosen based on the expected route of administration of the composition in therapeutic applications. The route of administration of the composition depends on the condition to be treated. For example, intravenous injection may be preferred for treatment of a systemic disorder such as a leukemia or lymphoma, and oral administration may be preferred to treat a gastrointestinal disorder such as a cancer of the gastrointestinal system, or an oral cancer. The route of administration and the dosage of the composition to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. Thus, depending on the condition, the composition can be administered orally, parenterally, intranasally, vaginally, rectally, lingually, sublingually, bucally, intrabuccaly and transdermally to the patient.

Accordingly, compositions designed for oral, lingual, sublingual, buccal and intrabuccal administration can be made without undue experimentation by means well known in the art, for example, with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these vanous compositions should be pharmaceutically pure and non-toxic in the amounts used.

The compositions of the present invention can be administered parenterally such as, for example, by intravenous, intramuscular, intrathecal or subcutaneous injection. Parenteral administration can be accomplished by incorporating the antibody compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the antibody composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

The present invention includes nasally administering to the mammal a therapeutically effective amount of the composition. As used herein, nasally administering or nasal administration includes administering the composition to the mucous membranes of the nasal passage or nasal cavity of the patient. As used herein, pharmaceutical compositions for nasal administration of a composition include therapeutically effective amounts of the agonist prepared by well-known methods to be administered, for example, as a nasal spray, nasal drop, suspension, gel, ointment, cream or powder. Administration of the composition may also take place using a nasal tampon or nasal sponge.

Administration of the pharmaceutical compositions of the invention the pharmaceutical compositions of the invention can be administered to animals, for example, humans in an amount sufficient to mount an immune response for the treatment or prevention of a disease, for example a viral disease or a bacterial disease (e.g., through vaccination, or through anti-bacterial or anti-viral therapy), or to slow the proliferation of cancer cells or to kill them entirely, it would be clear to those skilled in the art that the optimal schedule for administering such a pharmaceutical composition will vary based on the subject, the subjects height and weight and the severity of the disease. Ultimately, the use and schedule of administration of a pharmaceutical composition of the present invention will be decided by the treating physician, clinical protocols for determining dose range and scheduling are standard.

### EXAMPLE 1

### Materials and Methods

Cloning of HMGB1 and Production of HMGB1 B Box Mutants

The following methods were used to prepare clones and mutants of human HMGB 1. Recombinant full length human HMGB 1 (651 base pairs; GenBank Accession Number U51677) was cloned by PCR amplification from a human brain Quick-Clone cDNA preparation (Clontech, Palo Alto, Calif.) using the following primers; forward primer: 5′ GATGGGCAAAGGAGATCCTAAG 3′ (SEQ ID NO: 6) and reverse primer: 5′ GCGGCCGCTTATTCATCATCATCATCTTC 3′ (SEQ ID NO: 7). Human HMGB1 mutants were cloned and purified as follows. A truncated form of human HMGB 1 was cloned by PCR amplification from a Human Brain Quick-Clone cDNA preparation (Clontech, Palo Alto, Calif.). The primers used were (forward and reverse, respectively):

Carboxy terminus mutant (557 bp):

(SEQ ID NO: 8)

5′ GATGGGCAAAGGAGATCCTAAG 3′
and

(SEQ ID NO: 9)

5′ GCGGCCGC TCACTTGCTTTTTTCAGCCTTGAC 3′.
Amino terminus + B box mutant (486 bp):

(SEQ ID NO: 10)

5′ GAGCATAAGAAGAAGCACCCA 3′
and

(SEQ ID NO: 11)

5′ GCGGCCGC TCACTTGCTTTTTTCAGCCTTGAC 3′.
B box mutant (233 bp):

(SEQ ID NO: 12)

5′ AAGTTCAAGGATCCCAATGCAAAG 3′
and

(SEQ ID NO: 13)

5′ GCGGCCGCTCAATATGCAGCTATATCCTTTTC 3′.
Amino terminus + A box mutant (261 bp):

(SEQ ID NO: 13)

5′ GATGGGCAAAGGAGATCCTAAG 3′
and

(SEQ ID NO: 14)

5′ TCACTTTTTTGTCTCCCCTTTGGG 3′.

A stop codon was added to each mutant to ensure the accuracy of protein size. PCR products were subcloned into pCRII-TOPO vector EcoRI sites using the TA cloning method per manufacturer's instruction (Invitrogen, Carlsbad, Calif.). After amplification, the PCR product was digested with EcoRI and subcloned onto expression vector with a GST tag pGEX (Pharmacia); correct orientation and positive clones were confirmed by DNA sequencing on both strands. The recombinant plasmids were transformed into protease deficient E. Coli strains BL21 or BL21(DE3)plysS (Novagen, Madison, Wis.) and fusion protein expression was induced by isopropyl-D-thiogalactopyranoside (IPTG). Recombinant proteins were obtained using affinity purification with the glutathione Sepharose resin column (Pharmacia).

The HMGB mutants generated as described above have the following amino acid sequences:

Wild type HMGB 1:

(SEQ ID NO: 18)

MGKGDPKKPTGKMSSYAFFVQTCREEHKKKHPDASVNFSEFSKKCSERWK
TMSAKEKGKFEDMAKADKARYEREMKTYIPPKGETKKKFKDPNAPKRLPS
AFFLFCSEYRPKIKGEHPGLSIGDVAKKLGEMWNNTAADDKQPYEKKAAK
LKEKYEKDIAAYRAKGKPDAAKKGVVKAEKSKKKKEEEEDEEDEEDEEEE
EDEEDEEDEEEDDDDE
Carboxy terminus mutant:

(SEQ ID NO: 19)

MGKGDPKKPTGKMSSYAFFVQTCREEHKKKHPDASVNFSEFSKKCSERWK
TMSAKEKGKFEDMAKADKARYEREMKTYIPPKGETKKKFKDPNAPKRLPS
AFFLFCSEYRPKIKGEHPGLSIGDVAKKLGEMWNNTAADDKQPYEKKAAK
LKEKYEKDIAAYRAKGKPDAAKKGVVKAEKSK
B Box mutant:

(SEQ ID NO: 20)

FKDPNAPKRLPSAFFLFCSEYRPKIKGEHPGLSIGDVAKKLGEMWNNTAA
DDKQPYEKKAAKLKEKYEKDIAAY
Amino terminus + A Box mutant:

(SEQ ID NO: 21)

MGKGDPKKPTGKMSSYAFFVQTCREEHKKKHPDASVNFSEFSKKCSERWK
TMSAKEKGKFEDMAKADKARYEREMKTYIPPKGET,
wherein the A box consists of the sequence

(SEQ ID NO: 22)

PTGKMSSYAFFVQTCREEHKKKHPDASVNFSEFSKKCSERWKTMSAKEKG
KFEDMAKADKARYEREMKTYIPPKGET

A polypeptide generated from a GST vector lacking HMGB1 protein was included as a control (containing a GST tag only). To inactive the bacterial DNA that bound to the wild type HMGB 1 and some of the mutants (carboxy terminus and B box), DNase I (Life Technologies), for carboxy terminus and B box mutants, or benzonase nuclease (Novagen, Madison, Wis.), for wild type HMGB1, was added at about 20 units/ml bacteria lysate. Degradation of DNA was verified by ethidium bromide staining of the agarose gel containing HMGB1 proteins before and after the treatment. The protein eluates were passed over a polymyxin B column Pierce, Rockford, Ill.) to remove any contaminating LPS, and dialyzed extensively against phosphate buffered saline to remove excess reduced glutathione. The preparations were then lyophilized and redissolved in sterile water before use. LPS levels were less than 60 pg/μg protein for all the mutants and 300 pg/μg for wild type HMG-1 as measured by Limulus amebocyte lysate assay (Bio Whittaker Inc., Walkersville, Md.). The integrity of protein was verified by SDS-PAGE. Recombinant rat HMGB1 (Wang et al., Science 285: 248-251, 1999) was used in some experiments since it does not have degraded fragments as observed in purified human HMGB1.

Peptide Synthesis

Peptides were synthesized and HPLC purified at Utah State University Biotechnology Center (Logan, Utah) at 90% purity. Endotoxin was not detectable in the synthetic peptide preparations as measured by Limulus assay.

Cell Culture

Murine macrophage-like RAW 264.7 cells (American Type Culture Collection, Rockville, Md.) were cultured in RPMI 1640 medium (Life Technologies, Grand Island N.Y.) supplemented with 10% fetal bovine serum (Gemini, Catabasas, Calif.), penicillin and streptomycin (Life Technologies) and were used at 90% confluence in serum-free Opti-MEM I medium (Life Technologies, Grand Island, N.Y.). Polymyxin B (Sigma, St. Louis, Mo.) was routinely added at 100-1,000 units/ml to neutralize the activity of any contaminating LPS as previously described; polymyxin B alone did not influence cell viability assessed with trypan blue (Wang et al., supra). Polymyxin B was not used in experiments of synthetic peptide studies.

Measurement of TNF Release From Cells

TNF release was measured by a standard murine fibroblast L929 (ATCC, American Type Culture Collection, Rockville, Md.) cytotoxicity bioassay (Bianchi et al., Journal of Experimental Medicine 183:927-936, 1996) with the minimum detectable concentration of 30 pg/ml. Recombinant mouse TNF was obtained from R&D system Inc., (Minneapolis, MN). Murine fibroblast L929 cells (ATCC) were cultured in DMEM (Life Technologies, Grand Island, N.Y.) supplemented with 10% fetal bovine serum (Gemini, Catabasas, Calif.), penicillin (50 units/ml) and streptomycin (50 μg/ml) (Life Technologies) in a humidified incubator with 5% CO₂.

Antibody Production

Polyclonal antibodies against HMGB1 B box were raised in rabbits (Cocalico Biologicals, Inc., Reamstown, Pa.) and assayed for titer by immunoblotting. IgG was purified from anti-HMGB1 antiserum using Protein A agarose according to manufacturer's instructions (Pierce, Rockford, Ill.). Anti-HMGB1 B box antibodies were affinity purified by using cyanogen bromide activated Sepharose beads (Cocalico Biological, Inc.). Non-immune rabbit IgG was purchased from Sigma (St. Louis, Mo.). Antibodies detected full length HMGB1 and B box in immunoassay, but did not cross react with TNF, IL-1 and IL-6.

Animal Experiments

TNF knock out mice were obtained from Amgen (Thousand Oaks, Calif.) and were on a B6x129 background. Age-matched wild-type B6x129 mice were used as control for the studies. Mice were bred in-house at the University of Florida specific pathogen-free transgenic mouse facility (Gainesville, Fla.) and were used at 6-8 weeks of age.

Male 6-8 week old Balb/c and C3H/HeJ mice were purchased from Harlen Sprague-Dawley (Indianapolis, Ind.) and were allowed to acclimate for 7 days before use in experiments. All animals were housed in the North Shore University Hospital Animal Facility under standard temperature, and a light and dark cycle.

D-galactosamine Sensitized Mice

The D-galactosamine-sensitized model has been described previously (Galanos et al., Proc Natl. Acad. Sci. USA 76: 5939-5943, 1979; and Lehmann et al., J. Exp. Med. 165: 657663, 1997). Mice were injected intraperitoneally with 20 mg D-galactosamine-HCL (Sigma)/mouse (in 200 μl PBS) and 0.1 or 1 mg of either HMGB1 B box or vector protein (in 200 μl PBS). Mortality was recorded daily for up to 72 hours after injection; survivors were followed for 2 weeks, and no later deaths from B box toxicity were observed.

Statistical Analysis

Data are presented as mean±SEM unless otherwise stated. Differences between groups were determined by two-tailed Student's t-test, one-way ANOVA followed by the least significant difference test or 2 tailed Fisher's Exact Test.

### EXAMPLE 2

### Mapping the HMGB1 Domains for Promotion of Cytokine Activity

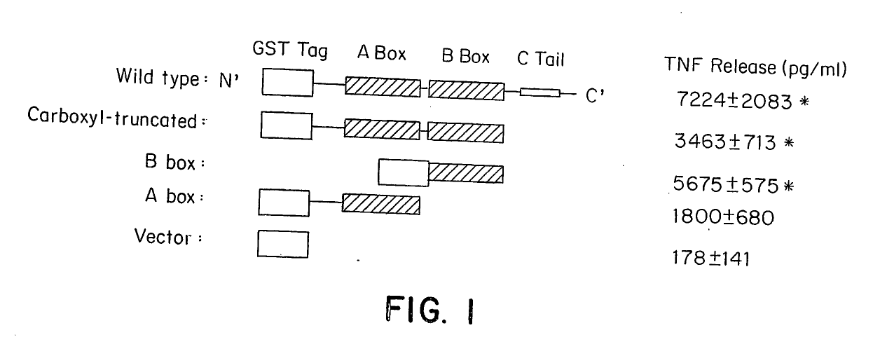

HMGB1 has 2 folded DNA binding domains (A and B boxes) and a negatively charged acidic carboxyl tail. To elucidate the structural basis of HMGB1 cytokine activity, and to map the inflammatory protein domain, full length and truncated forms of HMGB1 were expressed by mutagenesis and the purified proteins were screened for stimulating activity in monocyte cultures (FIG. 1). Full length HMGB1, a mutant in which the carboxy terminus was deleted, a mutant containing only the B box, and a mutant containing only the A box were generated. These mutants of human HMGB1 were made by polymerase chain reaction (PCR) using specific primers as described herein, and the mutant proteins were expressed using a glutathione S-transferase (GST) gene fusion system (Pharmacia Biotech, Piscataway, N.J.) in accordance with the manufacturer's instructions. Briefly, DNA fragments, made by PCR methods, were fused to GST fusion vectors and amplified in E. coli. The expressed HMGB1 protein and HMGB1 mutants and were then isolated using GST affinity column.

The effect of the mutants on TNF release from Murine macrophage-like RAW 264.7 cells (ATCC) was carried out as follows. RAW 264.7 cells were cultured in RPMI 1640 medium (Life Technologies, Grand Island N.Y.) supplemented with 10% fetal bovine serum (Gemini, Catabasas, Calif.), penicillin and streptomycin (Life Technologies). Polymyxin (Sigma, St. Louis, Mo.) was added at 100 units/ml to suppress the activity of any contaminating LPS. Cells were incubated with 1 μg/ml of full length (wild-type) HMGB1 and each HMGB1 mutant protein in Opti-MEM I medium for 8 hours, and conditioned supernatants (containing TNF which had been released from the cells) were collected and TNF released from the cells was measured by a standard murine fibroblast L929 (ATCC) cytotoxicity bioassay (Bianchi et al., supra) with the minimum detectable concentration of 30 pg/ml. Recombinant mouse TNF was obtained from R & D Systems Inc., (Minneapolis, Minn.) and used as control in these experiments. The results of this study are shown in FIG. 1. Data in FIG. 1 are all presented as mean+SEM unless otherwise indicated. (=6−10).

As shown in FIG. 1, wild-type HMGB1 and carboxyl-truncated HMGB1 significantly stimulated TNF release by monocyte cultures (murine macrophage-like RAW 264.7 cells). The B box was a potent activator of monocyte TNF release. This stimulating effect of the B box was specific, because A box only weakly activated TNF release.

### EXAMPLE 3

### HMGB1 B Box Protein Promotes Cytokine Activity in a Dose Dependent Manner

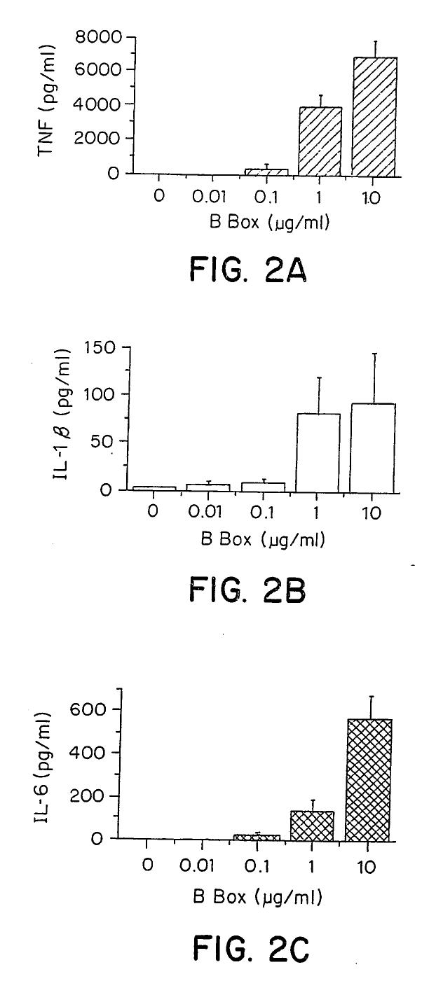

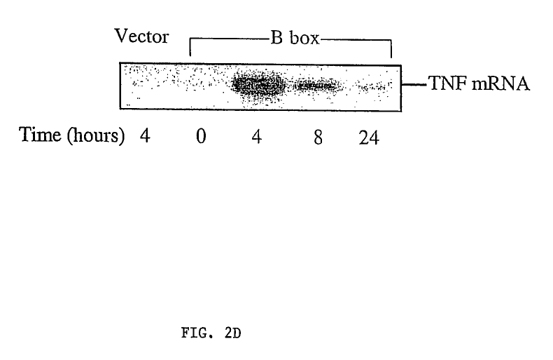

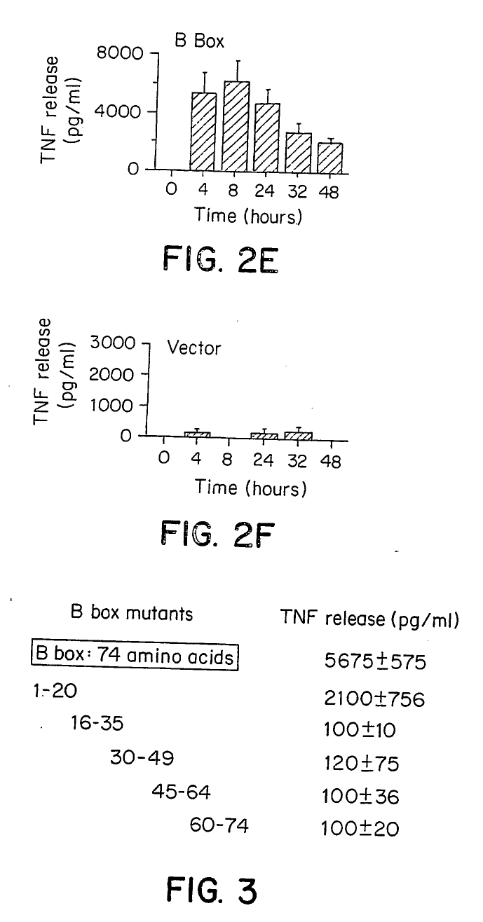

To further examine the effect of HMGB1 B box on cytokine production, varying amounts of HMGB1 B box were evaluated for the effects on TNF, IL-1B, and IL-6 production in murine macrophage-like RAW 264.7 cells. RAW 264.7 cells were stimulated with B box protein at 0-10 μg/ml, as indicated in FIGS. 2A-2C for 8 hours. Conditioned media were harvested and measured for TNF, IL-1β and IL-6 levels. TNF levels were measured as described herein, and IL-1β and IL-6 levels were measured using the mouse IL-1β and IL-6 enzyme-linked immunosorbent assay (ELISA) kits (R&D System Inc., Minneapolis, 1) and N>5 for all experiments. The results of the studies are shown in FIGS. 2A-2C.

As shown in FIG. 2A, PNF release from RAW 264.7 cells increased with increased amounts of B box administered to the cells. As shown in FIG. 2B, addition of 1 μg/ml or 10 μg/ml of B box resulted in increased release of IL-1β from RAW 264.7 cells. In addition, as shown in FIG. 2C, IL-6 release from RAW 264.7 cells increased with increased amounts of B box administered to the cells.

The kinetics of B box-induced TNF release was also examined. TNF release and TNF mRNA expression was measured in RAW 264.7 cells induced by B box polypeptide or GST tag polypeptide only used as a control (vector) (10 μg/ml) for 0 to 48 hours. Supernatants were analyzed for TNF protein levels by an L929 cytotoxicity assay (N=3-5) as described herein. For mRNA measurement, cells were plated in 100 mm plate and treated in Opti-MEM I medium containing B box polypeptide or the vector alone for 0, 4, 8, or 24 hours, as indicated in FIG. 2D. The vector only sample was assayed at the 4 hour time point. Cells were scraped off the plate and total RNA was isolated by RNAzol B method in accordance with the manufacturer's instructions (Tel-Test “B”, Inc., Friendswood, Tex.). TNF (287 bp) was measured by RNase protection assay (Ambion, Austin, Tex.). Equal loading and the integrity of RNA was verified by ethidium bromide staining of the RNA sample on agarose-formaldehyde gel. The results of the RNase protection assay are shown in FIG. 2D. As shown in FIG. 2D, B box activation of monocytes occurred at the level of gene transcription, because TNF mRNA was increased significantly in monocytes exposed to B box protein (FIG. 2B). TNF mRNA expression was maximal at 4 hours and decreased at 8 and 24 hours. The vector only control (GST tag) showed no effect on TNF mRNA expression. A similar study was carried out measuring TNF protein released from RAW 264.7 cells 0, 4, 8, 24, 32 or 48 hours after administration of B box or vector only (GST tag), using the L929 cytotoxicity assay described herein. Compared to the control (medium only), B box treatment stimulated TNF protein expression (FIG. 2F) and vector alone (FIG. 2E) did not. Data are representative of three separate experiments. Together these data indicate that the HMGB1 B box domain has cytokine activity and is responsible for the cytokine stimulating activity of full length HMGB1.

In summary, the HMGB1 B box dose-dependently stimulated release of TNF, IL-1β and IL-6 from monocyte cultures (FIGS. 2A-2C), in agreement with the inflammatory activity of full length HMGB1 (Andersson et al., J. Exp. Med. 192: 565-570, 2000). In addition, these studies indicate that maximum TNF protein release occurred within 8 hours (FIG. 2F). This delayed pattern of TNF release is similar to TNF release induced by HMGB31 itself, and is significantly later than the kinetics of TNF induced by LPS (Andersson et al., supra).

### EXAMPLE 4

### The First 20 Amino Acids of the HMGB1 B Box Stimulate TNF Activity

The TNF-stimulating activity of the HMGB1 B box was further mapped. This study was carried out as follows. Fragments of the B box were generated using synthetic peptide protection techniques, as described herein. Five HMGB 1 B box fragments (from SEQ ID NO: 20), containing amino acids 1-20, 16-25, 30-49, 45-64, or 60-74 of the HMGB1 B box were generated, as indicated in FIG. 3. RAW 264.7 cells were treated with B box (1 μg/ml) or a synthetic peptide fragment of the B box (10 μg/ml), as indicated in FIG. 3 for 10 hours and TNF release in the supernatants was measured as described herein. Data shown are mean±SEM, (n=3 experiments, each done in duplicate and validated using 3 separate lots of synthetic peptides). As shown in FIG. 3, TNF-stimulating activity was retained by a synthetic peptide corresponding to amino acids 1-20 of the HMGB1 B box of SEQ ID NO: 20 (fkdpnapkrlpsafflfcse; SEQ ID NO: 23). The TNF stimulating activity of the 1-20-mer was less potent than either the full length synthetic B box (1-74-mer), or fall length HMGB1, but the stimulatory effects were specific because the synthetic 20-mers for amino acid fragments containing 16-25, 30-49, 45-64, or 60-74 of the HMGB 1 B box did not induce TNF release. These results are direct evidence that the macrophage stimulating activity of the B box specifically maps to the first 20 amino acids of the HMGB B box domain of SEQ ID NO: 20). This B box fragment can be used in the same manner as a polypeptide encoding a full length B box polypeptide, for example, to stimulate releases of a proinflammatory cytokine, or to treat a condition in a patient characterized by activation of an inflammatory cytokine cascade.

### EXAMPLE 5

### HMGB1 B Box Protein is Toxic to D-galactosamine-sensitized Balb/c Mice

To investigate whether the HMGB1 B box has cytokine activity in vivo, we administered HMGB1 B box protein to unanesthetized Balb/c mice sensitized with D-galactosamine (D-gal), a model that is widely used to study cytokine toxicity (Galanos et al., supra). Briefly, mice (20-25 gram, male, Harlan Sprague-Dawley, Indianapolis, IN) were intraperitoneally injected with D-gal (20 mg) (Sigma) and B box (0.1 mg/ml/mouse or 1 mg/ml/mouse) or GST tag (vector; 0.1 mg/ml/mouse or 1 mg/ml/mouse), as indicated in Table 1. Survival of the mice was monitored up to 7 days to ensure no late death occurred. The results of this study are shown in Table 1.

TABLE 1
Toxicity of HMGB1 B box on D-galactosamine-sensitized Balb/c Mice

| | Treatment | Alive/total |
|---|---|---|
| Control | — | 10/10 |
| Vector | 0.1 mg/mouse | 2/2 |
| | 1 mg/mouse | 3/3 |
| B box | 0.1 mg/mouse | 6/6 |
| | 1 mg/mouse | 2/8* |

P < 0.01 versus vector alone as tested by Fisher's Exact Test

The results of this study showed that the HMGB1 B box was lethal to D-galactosamine-sensitized mice in a dose-dependent manner. In all instances in which death occurred, it occurred within 12 hours. Lethality was not observed in mice treated with comparable preparations of the purified GST vector protein devoid of B box.

### EXAMPLE 6

### Histology of D-Galactosamine-Sensitized Balb/c Mice or C3H/HeJ Mice Administered HMGB1 B Box Protein

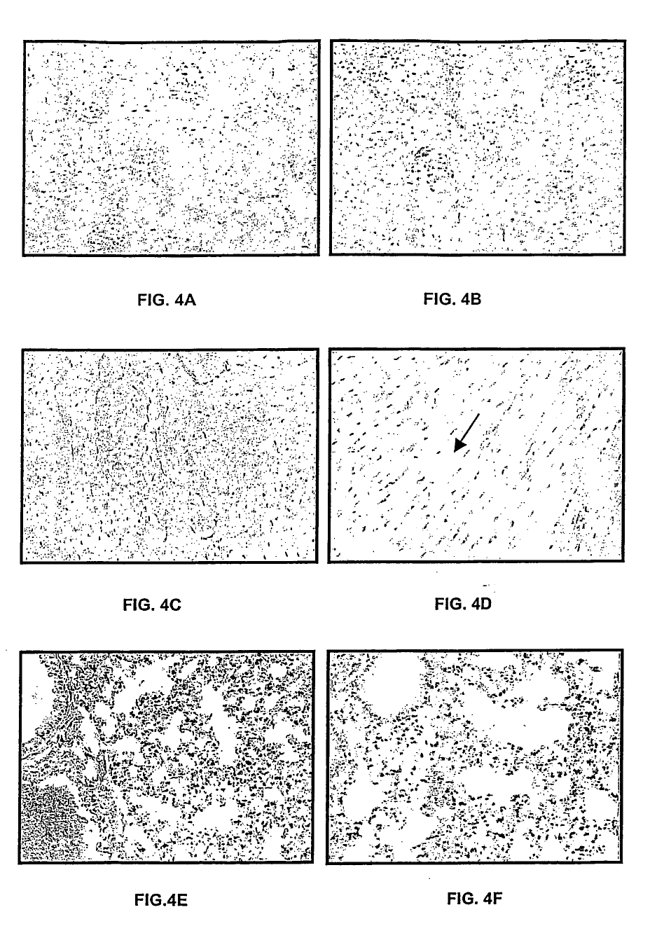

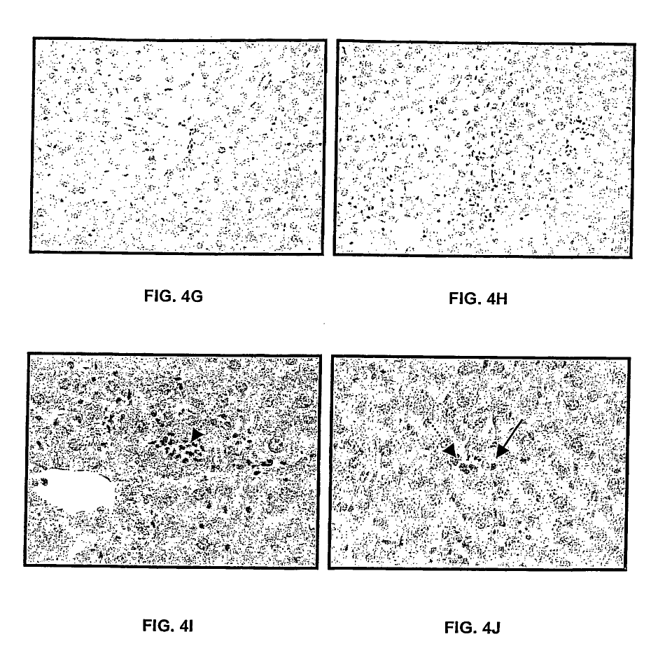

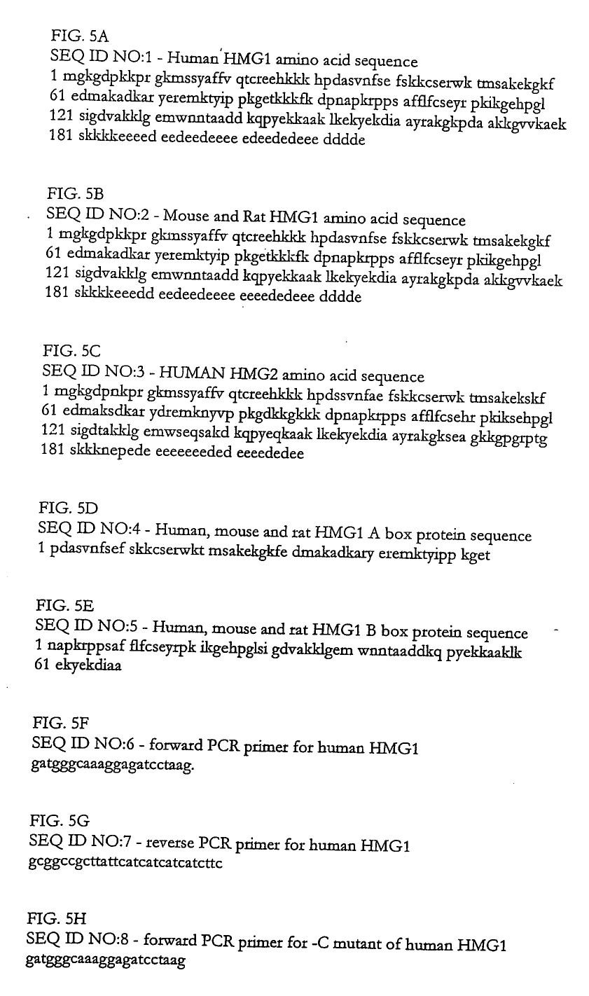

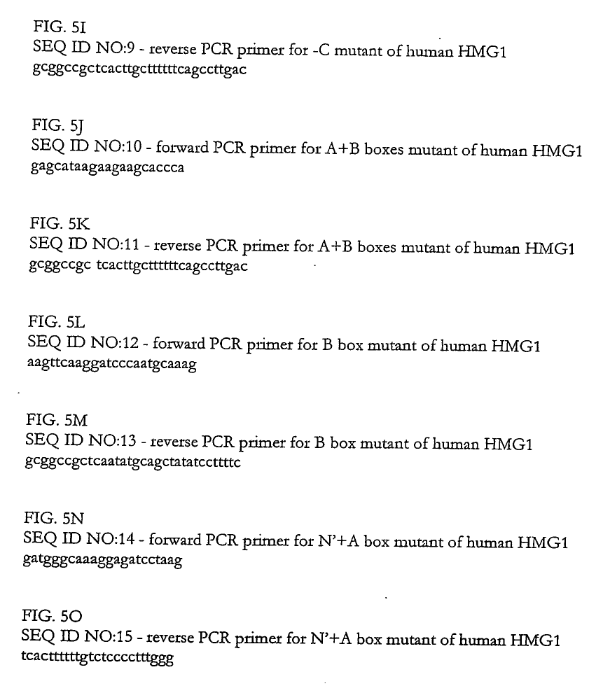

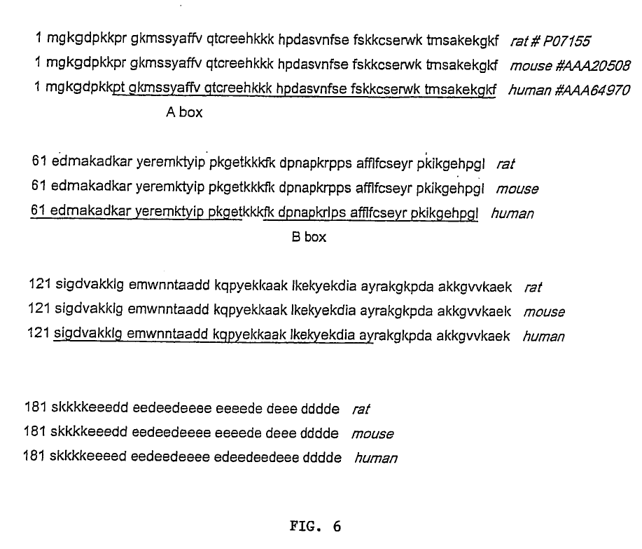

To further assess the lethality of the HMGB1 B box protein in vivo the HMGB1 B box was again administered to D-galactosamine-sensitized Balb/c mice. Mice (3 per group) received D-gal (20 mg/mouse) plus B box or vector (1 mg/mouse) intraperitoneally for 7 hours and were then sacrificed by decapitation. Blood was collected, and organs (liver, heart, kidney and lung) were harvested and fixed in 10% formaldehyde. Tissue sections were prepared with hematoxylin and eosin staining for histological evaluation (Criterion Inc., Vancouver, Canada). The results of these studies are shown in FIGS. 4A-4J, which are scanned images of hematoxylin and eosin stained kidney sections (FIG. 4A), myocardium sections (FIG. 4C), lung sections (FIG. 4E), and liver sections (FIGS. 4G and 41 obtained from an untreated mouse and kidney sections (FIG. 4B), myocardium sections (FIG. 4D), lung sections (FIG. 4F), and liver sections (FIGS. 4H and 4J) obtained from mice treated with the HMGB1 B box. Compared to the control mice, B box treatment caused no abnormality in kidneys (FIGS. 4A and 4B) and lungs (FIGS. 4E and 4F). The mice had some ischemic changes and loss of cross striation in myocardial fibers in the heart (FIGS. 4C and 4D as indicated by the arrow in FIG. 4D). Liver showed most of the damage by the B box as illustrated by active hepatitis (FIGS. 4G-4J). In FIG. 4J, hepatocyte dropouts are seen surrounded by accumulated polymorphonuclear leukocytes. The arrows in FIG. 4J point to the sites of polymorphonuclear accumulation (dotted) or apoptotic hepatocytes (solid). Administration of HMGB1 B box in vivo also stimulated significantly increased serum levels of IL-6 (315+93 vs. 20+7 pg/ml, B box vs. control, p<0.05) and IL-1β (15+3 vs. 4+1 pg/ml, B box vs. control, p<0.05).

Administration of B box protein to C3H/HeJ mice (which do not respond to endotoxin) was also lethal, indicating that HMGB1 B box is lethal in the absence of LPS signal transduction. Hematoxylin and eosin stained sections of lung and kidney collected 8 hours after administration of B box revealed no abnormal morphologic changes. Examination of sections from the heart however, revealed evidence of ischemia with loss of cross striation associated with amorphous pink cytoplasm in myocardial fibers. Sections from liver showed mild acute inflammatory responses, with some hepatocyte dropout and apoptosis, and occasional polymorphonuclear leukocytes. These specific pathological changes were comparable to those observed after administration of full length HMGB1 and confirm that the B box alone can recapitulate the lethal pathological response to HMGB1 in vivo.

To address whether the TNF-stimulating activity of HMGB1 contributes to the mediation of lethality by B box, we measured lethality in TNF knock-out mice (TNF-KO, Nowak et al., Am. J. Physiol. Regul. Integr. Comp. Physiol. 278: R1202-R1209, 2000) and the wild-type controls (B6x129 strain) sensitized with D-galactosamine (20 mg/mouse) and exposed to B box (1 mg/mouse, injected intraperitoneally). The B box was highly lethal to the wild-type mice (6 dead out of nine exposed) but lethality was not observed in the TNF-KO mice treated with B box (0 dead out of 9 exposed, p<0.05 v. wild type). Together with the data from the RAW 264.7 macrophage cultures, described herein, these data now indicate that the B box of HMGB1 confers specific TNF-stimulating cytokine activity.

While this invention has been particularly shown and described with references to preferred embodiments thereof, it will be understood by those skilled in the art that various changes in form and details may be made therein without departing from the scope of the invention encompassed by the appended claims.

What is claimed is:

1. A pharmaceutical composition comprising a polypeptide comprising an HMGB B box or a functional variant thereof, in an amount sufficient to treat a disease or condition by increasing an immune response in an individual administered said pharmaceutical composition.

2. The pharmaceutical composition of claim 1, wherein said HMGB B box is mammalian.

3. The pharmaceutical composition of claim 2, wherein said HMGB B box is human.

4. The pharmaceutical composition of claim 3, wherein said polypeptide comprises an HGB1 B box polypeptide.

5. The pharmaceutical composition of claim 4, wherein said polypeptide consists of an HMGB1 B box polypeptide.

6. The pharmaceutical composition of claim 1, further comprising a vaccine.

7. The pharmaceutical composition of claim 6, further comprising an adjuvant.

8. The pharmaceutical composition of claim 7, wherein said adjuvant is selected from the group consisting of one or more immunostimulatory oligonucleotides, an imidazoquinoline, monophosphoryl lipid A, Brad detoxified lipopolysaccharide.

9. The pharmaceutical composition of claim 8, wherein said immunostimulatory oligonucleotides comprise unmethylated CpG sequences.

10. An antibody attached to a polypeptide comprising an HMGB B box or a functional variant there of.

11. The antibody of claim 10, wherein said HMGB B box is mammalian.

12. The antibody of claim 11, wherein said HMGB B box is human.

13. The antibody of claim 12, wherein said polypeptide comprises an HMGB1 B box polypeptide.

14. The antibody of claim 13, wherein said polypeptide consists of an HMGB1 B box polypeptide.

15. The antibody of claim 10, wherein said antibody binds a rumor-associated polypeptide.

16. The antibody of claim 10, wherein said antibody is in a pharmaceutically acceptable carrier.

17. A method of simulating or increasing an immune response in an individual in need of immunostimulation, said method comprising administering to said individual a polypeptide comprising an HMGB B box or a functional variant thereof, in a amount sufficient to stimulate or increase said immune response.

18. The method of claim 17, wherein said individual is being treated for cancer.

19. The method of claim 17, wherein said HMGB B box is mammalian.

20. The method of claim 19, wherein said HMGB B box is human.

21. The method of claim 20, wherein said polypeptide comprises an HMGB1 B box.

22. The method of claim 21, wherein said polypeptide consists of an HMGB1 B box.

23. The method of claim 17, wherein said polypeptide is co-administered with a vaccine.

24. The method of claim 23, wherein said polypeptide is co-administered with a m further adjuvant.

25. The method of claim 24, wherein said adjuvant is selected from the group consisting of one or more immunostimulatory oligoneucleotides, an imidazoquinoline, monophosphoryl lipid A, and detoxified W lipopolysaccharide.

26. The method of claim 25, wherein said immunostimulatory oligonucleotides comprise unmethylated CpG sequences.

27. The method of claim 17, wherein said administration is systemic.

28. The method of claim 17, wherein said administration is localized to a target site.

29. The method of claim 17, wherein said polypeptide is attached to an antibody specific to a target site in the individual in need of immunostimulation.

30. The method of claim 17, wherein said polypeptide is in a pharmaceutically acceptable carrier.

31. A method of treating cancer in an individual, said method comprising administering to said individual a therapeutically effective amount of a polypeptide comprising an HMGB B box or a functional variant thereof.

32. The method of claim 31, wherein said HMGB B box is mammalian.

33. The method of claim 32, wherein said HMGB B box is human.

34. The method of claim 33, wherein said polypeptide comprises an HMGB1 B box polypeptide.

35. The method of claim 34, wherein said polypeptide consists of an HMGB1 B box polypeptide.

36. The method of claim 31, wherein said polypeptide is co-administered with a vaccine.

37. The method of claim 36, wherein said polypeptide is co-administered with a further adjuvant.

38. The method of claim 37, wherein said adjuvant is selected from the group consisting of one or more immunostimulatory oligonucleotides, an imidazoquinoline monophosphoryl lipid A, and detoxified lipopolysacharide.

39. The method of claim 38, wherein said immunostimulatory oligonucleotides comprise unmethylated CpG sequences.

40. The method of claim 31, wherein said administration is systemic.

41. The method of claim 31, wherein said administration is localized to a target site.

42. The method of claim 41, wherein said target site is a tumor.

43. The method of claim 31, wherein said polypeptide is attached to an antibody.

44. The method of claim 43, wherein said antibody binds a tumor-associated polypeptide.

45. The method of claim 31, wherein said polypeptide is in a pharmaceutically acceptable carrier.
